(19) United States
(12) Patent Application Publication
(10) Pub. No.: US 2021/0195907 A1
(43) Pub. Date: Jul. 1, 2021

(54) PROCESS FOR PRODUCING AN IMPROVED FERMENTED MILK PRODUCT USING A SPORULATION NEGATIVE BACILLUS STRAIN

(71) Applicant: Chr. Hansen A/S, Hoersholm (DK)

(72) Inventors: Mette Dines CANTOR, Hoersholm (DK); Karin BJERRE, Hoersholm (DK); Elahe Ghanei MOGHADAM, Hoersholm (DK); Helle Skov GULDAGER, Hoersholm (DK); Patrick DERKX, Hoersholm (DK); Patricia Dominguez CUEVAS, Hoersholm (DK)

(73) Assignee: Chr. Hansen A/S, Hoersholm (DK)

(21) Appl. No.: 17/269,581

(22) PCT Filed: Aug. 20, 2019
(86) PCT No.: PCT/EP2019/072249

§ 371 Date: Feb. 19, 2021

(30) Foreign Application Priority Data
Aug. 21, 2018 (EP) 18189920.4

(51) Int. Cl. A23C 9/127; A23C 13/16; C12R 1/125; C12N 1/20

(52) U.S. Cl. CPC A23C 9/127; A23C 13/16; C12R 1/125; A23Y 2220/15; A23Y 2240/41; A23Y 2240/21; A23Y 2240/75; C12N 1/20

(57) ABSTRACT

The invention relates to a method for producing a fermented dairy product, comprising: (a) providing a milk substrate, (b) fermenting said milk substrate with a lactic acid bacterium starter culture, wherein step (b) is conducted in the presence of at least one Bacillus strain selected from the group consisting of a sporulation-negative Bacillus subtilis subsp. natto strain, a sporulation-negative Bacillus coagulans strain, wherein a sporulation-negative strain is a strain, which forms no spores when subjected to the following method: i) inoculating 1% of a culture of the strain to be tested, grown over night in Veal Infusion Broth (VIB) at 37° C., 180 rpm, into 50 ml of a standard sporulation inducing medium contained in a 500 ml baffled shake flask, ii) allowing the inoculated medium to grow overnight at 37° C. while subjecting it to shaking at 0rpm, and iii) testing for spores the next day.

### FIELD OF INVENTION

The present invention relates to a method for producing a fermented dairy product, comprising:
(a) providing a milk substrate,
(b) fermenting said milk substrate with a lactic acid bacterium starter culture.

### BACKGROUND OF INVENTION

The food industry uses numerous different types of bacteria for preparing food items. For the preparation of fermented dairy products, such as yogurts, cheese or buttermilk, lactic acid bacteria (LAB) are most commonly used. LAB and their metabolic products significantly contribute to the taste and texture of fermented products and inhibit food spoilage by producing considerable amounts of lactic acid.

LABs strains that are currently used by the food industry for preparing fermented dairy products originate from different taxonomical groups, e.g. the genera Streptococcus, Lactococcus, Lactobacillus, Leuconostoc, and Bifidobacterium. The ability of the strains used for fermentation to confer texture to dairy products is to some extent linked to the production of polysaccharides. Not every strain which has been found to have particularly suitable fermentation characteristics, e.g. a good acidification profile, also has good texturizing characteristics. Therefore, it is often required to improve the texture of fermented dairy products.

Different approaches have been used in the prior art for increasing the texture of such products. For example, additives such as gelatin, pectins, alginates, carboxymethyl cellulose, gums, starch, and fiber can be added to the product after its production [1]. However, such additives are generally undesirable in view of the increasing consumer demand for “clean label” products.

Yet another approach for increasing texture is focused on the optimization of the LAB strains used in the fermentation. For example, the use of genetically modified strains with increased galactokinase activity was found to have a significant impact on the texture of products produced with such strains [2]. While these modified strains are highly effective, a high number of consumers tend to prefer naturally occurring strains in dairy products.

The co-fermentation of LABs with bacteria that do not belong to the group of LAB has so far not attracted much attention in the dairy industry. One reason for this resides in the fact that LAB produce high amounts of lactic acid during fermentation which results in a considerable reduction of the pH to 4-5 during fermentation. Most bacteria tolerate only moderate pH reductions which makes them unsuitable for being used in the preparation of fermented dairy products.

Bacteria of the genus Bacillus are not commonly used for dairy fermentation. Nevertheless, there is some evidence that Bacillus strains have been employed in the past for preparing dairy products, such as yogurt. Reference [3] describes the use of Bacillus strains for fermenting milk products such as yogurt in the absence of classical LAB starter cultures.

Reference [4] describes the use of a Bacillus subtilis strain for producing a fermented milk product that might be of therapeutic value. It is reported that antibacterial substances produced by the Bacillus strain provide for a product with long shelf-life and putative therapeutic properties.

Reference [5] describes a method for producing fermented milk using Bacillus subtilis. The method comprises two successive steps. In a first step, milk is fermented with Bacillus subtilis for several hours. In this step, the proteins in the milk are degraded into amino acids or oligo-peptides by Bacillus proteases. Subsequently, LAB are added to the milk and the fermentation is continued until the desired pH is reached.

Reference [6] discloses a method for preparing yogurt using levansucrase-producing strains of Bacillus licheniformis or Bacillus subtilis.

Reference [7] describes the preparation of fermented milk products with cheese flavor using a combination of Streptococcus thermophilus and Bacillus stereothermophilus to obtain a product with a cheesy flavor.

Reference [8] is an international patent application which discloses co-fermentation of Streptococcus thermophilus with different Bacillus strains, such as Bacillus subtilis subsp. natto for preparing a thermophilic fermented dairy product.

Finally, Reference [9] reports experiments in which potential effects of contamination by Bacillus subtilis and Bacillus licheniformis on the rheological and textural properties of sour cream have been analyzed.

### SUMMARY OF INVENTION

The present invention relates to a method for producing a fermented dairy product, comprising:
(a) providing a milk substrate,
(b) fermenting said milk substrate with a lactic acid bacterium starter culture,
   wherein step (b) is conducted in the presence of at least one Bacillus strain selected from the group consisting of a sporulation-negative Bacillus subtilis subsp. natto strain, a sporulation-negative Bacillus coagulans strain, wherein a sporulation-negative strain is a strain, which forms no spores when subjected to the following method:

i) inoculating 1% of a culture of the strain to be tested, grown over night in Veal Infusion Broth (VIB) at 37° C., 180 rpm, into 50 ml of a standard sporulation inducing medium contained in a 500 ml baffled shake flask,

ii) allowing the inoculated medium to grow overnight at 37° C. while subjecting it to shaking at 200 rpm, and

iii) testing for spores the next day.

A major obstacle in using Bacillus strains in production of fermented dairy products is that many Bacillus strains sporulate, which is highly undesired in industrial production of fermented dairy products. For this reason alone, the use of Bacillus strains in production of fermented dairy products has been avoided up to now. However, the present invention has now shown that it is possible to produce sporulation-negative Bacillus strains from sporulation-positive Bacillus mother strains with strong texturizing capacity, which maintain or even improves the texturizing capacity of the mother strain.

Also, it has now been surprisingly found that the texture of fermented dairy products can be significantly increased when fermenting a milk substrate with a lactic acid bacterium starter culture in the presence of at least one sporulation-negative Bacillus subtilis subsp. natto or at least one sporulation-negative Bacillus coagulans strain. It has been found that the said strains improve the texture of both thermophilic fermented milk products, such as yogurt, as well as that of mesophilic fermented milk products, such as sour creams. In particular, the said strains improve the texture as measured by shear stress and/or gel stiffness.

It appears that sporulation-negative mutants of the strains of these Bacillus species improve the texture conferred to the dairy product by the LAB. The mechanism by which the Bacillus strains exert this effect is unknown. Notably, the shear stress and gel stiffness of products manufactured by the method of the invention is very high and, in some cases, reaches a level which is fourfold higher than the corresponding shear stress achieved by the same LAB starter culture without the Bacillus strain. In addition, the fermentation of the milk substrate with LAB in the presence of Bacillus has been shown herein to significantly reduce the time that is required for reaching a target pH of e.g. 4.5. To this extent, the method of the invention aids in the reduction of costs involved with the production process.

Notably, the shear stress and gel stiffness of products manufactured by the method of the invention is very high and, in some cases, higher than the corresponding shear stress and gel stiffness achieved by the corresponding sporulation-positive Bacillus mother strain. Likewise, the acidification time in the method of the invention is short and, in some cases, shorter than the corresponding acidification time achieved by the corresponding sporulation-positive Bacillus mother strain.

In accordance with the above surprising findings, strains of sporulation-negative mutants of the species Bacillus subtilis subsp. natto or Bacillus coagulans may be used as additives to common mesophilic and thermophilic LAB starter cultures for improving the texture of fermented dairy products, e.g. by increasing shear stress or gel stiffness (Complex Modulus). The present invention provides novel fermentation methods using LAB strains and strains of sporulation-negative mutants of Bacillus subtilis subsp. natto or Bacillus coagulans as well as starter cultures comprising the respective combination of strains. Finally, the present invention provides novel sporulation-negative strains of Bacillus subtilis subsp. natto or Bacillus coagulans.

It is yet not completely understood how the Bacillus species influence the texturizing properties of the lactic acid bacteria. It however appears that the Bacillus strains do not propagate during fermentation to a significant extent. However, it has been shown herein that a significant growth of the Bacillus strains is not required for exerting the positive influence on LAB fermentation.

The present invention further relates to a Bacillus strain selected from the group consisting of a Bacillus subtilis subsp. natto and a Bacillus coagulans strain, which is a sporulation-negative mutant of a sporulation-positive mother strain, wherein a sporulation-negative strain is a strain, which forms no spores when subjected to the following method:

i) inoculating 1% of a culture of the strain to be tested, grown over night in Veal Infusion Broth (VIB) at 37° C., 180 rpm, into 50 ml of a standard sporulation inducing medium contained in a 500 ml baffled shake flask,

ii) allowing the inoculated medium to grow overnight at 37° C. while subjecting it to shaking at 200 rpm, and

iii) testing for spores the next day.

The present invention further relates to a composition for producing a fermented dairy product, comprising
(a) a lactic acid bacterium starter culture, and
(b) a Bacillus strain selected from the group consisting of a sporulation-negative Bacillus subtilis subsp. natto strain and a sporulation-negative Bacillus coagulans strain, and wherein a sporulation-negative strain is a strain, which forms no spores when subjected to the following method:

i) inoculating 1% of a culture of the strain to be tested, grown over night in Veal Infusion Broth (VIB) at 37° C., 180 rpm, into 50 ml of a standard sporulation inducing medium contained in a 500 ml baffled shake flask,

ii) allowing the inoculated medium to grow overnight at 37° C. while subjecting it to shaking at 200 rpm, and

iii) testing for spores the next day.

The present invention further relates to a fermented dairy product obtainable by the method of the invention.

The present invention further relates to A fermented dairy product, comprising
(a) a lactic acid bacterium starter culture, and
(b) a Bacillus strain selected from the group consisting of a sporulation-negative Bacillus subtilis subsp. natto strain and a sporulation-negative Bacillus coagulans strain, and wherein a sporulation-negative strain is a strain, which forms no spores when subjected to the following method:

i) inoculating 1% of a culture of the strain to be tested, grown over night in Veal Infusion Broth (VIB) at 37° C., 180 rpm, into 50 ml of a standard sporulation inducing medium contained in a 500 ml baffled shake flask,

ii) allowing the inoculated medium to grow overnight at 37° C. while subjecting it to shaking at 200 rpm, and

iii) testing for spores the next day.

The present invention further relates to a Use of a Bacillus strain selected from the group consisting of a sporulation-negative Bacillus subtilis subsp. natto strain and a sporulation-negative Bacillus coagulans strain for increasing the shear stress, gel stiffness and/or gel firmness of a mesophilic fermented dairy product, and wherein a sporulation-negative strain is a strain, which forms no spores when subjected to the following method:

i) inoculating 1% of a culture of the strain to be tested, grown over night in Veal Infusion Broth (VIB) at 37° C., 180 rpm, into 50 ml of a standard sporulation inducing medium contained in a 500 ml baffled shake flask,

ii) allowing the inoculated medium to grow overnight at 37° C. while subjecting it to shaking at 200 rpm, and

iii) testing for spores the next day.

Definitions

The term “thermophile” herein refers to microorganisms that thrive best at temperatures above 35° C. The industrially most useful thermophilic bacteria include Streptococcus spp. and Lactobacillus spp. The term “thermophilic fermentation” herein refers to fermentation at a temperature above about 35° C., such as between about 35° C. to about 45° C. The term “thermophilic fermented dairy product” refers to fermented milk products prepared by thermophilic fermentation of a thermophilic starter culture and include such fermented milk products as set-yoghurt, stirred-yoghurt and drinking yoghurt, e.g. Yakult.

The term “mesophile” herein refers to microorganisms that thrive best at moderate temperatures (15° C.-35° C.). The industrially most useful mesophilic bacteria include Lactococcus spp. and Leuconostoc spp. The term “mesophilic fermentation” herein refers to fermentation at a temperature between about 22° C. and about 35° C. The term “mesophilic fermented dairy product” refers to fermented milk products prepared by mesophilic fermentation of a mesophilic starter culture and include such fermented milk products as buttermilk, sour milk, cultured milk, smetana, sour cream, Kefir and fresh cheese, such as quark, tvarog and cream cheese.

The term “milk” is to be understood as the lacteal secretion obtained by milking any mammal, such as cows, sheep, goats, buffaloes or camels. In a preferred embodiment, the milk is cow's milk. The term milk also includes protein/fat solutions made of plant materials, e.g. soy milk.

The term “milk substrate” may be any raw and/or processed milk material that can be subjected to fermentation according to the method of the invention. Thus, useful milk substrates include, but are not limited to, solutions/suspensions of any milk or milk-like products comprising protein, such as whole or low fat milk, skim milk, buttermilk, reconstituted milk powder, condensed milk, dried milk, whey, whey permeate, lactose, mother liquid from crystallization of lactose, whey protein concentrate, or cream. Obviously, the milk substrate may originate from any mammal, e.g. being substantially pure mammalian milk, or reconstituted milk powder.

Prior to fermentation, the milk substrate may be homogenized and pasteurized according to methods known in the art.

“Homogenizing” as used herein means intensive mixing to obtain a soluble suspension or emulsion. If homogenization is performed prior to fermentation, it may be performed so as to break up the milk fat into smaller sizes so that it no longer separates from the milk. This may be accomplished by forcing the milk at high pressure through small orifices.

“Pasteurizing” as used herein means treatment of the milk substrate to reduce or eliminate the presence of live organisms, such as microorganisms. Preferably, pasteurization is attained by maintaining a specified temperature for a specified period of time. The specified temperature is usually attained by heating. The temperature and duration may be selected in order to kill or inactivate certain bacteria, such as harmful bacteria. A rapid cooling step may follow. “Fermentation” in the methods of the present invention means the conversion of carbohydrates into alcohols or acids through the action of a microorganism. Preferably, fermentation in the methods of the invention comprises conversion of lactose to lactic acid.

The expression “acidification time” means the period of time from the start of the fermentation to the target pH has been reached.

### DETAILED DISCLOSURE

The invention provides a novel method of manufacturing a dairy product which is based on the fermentation of a substrate with LAB in the presence of a sporulation-negative Bacillus strain.

Bacillus Strain

The sporulation-negative strain of the invention may be obtained using as a mother strain any Bacillus strain selected from group of any Bacillus subtilis subsp. natto strain and any sporulation-negative Bacillus coagulans strain.

In a particular embodiment of the invention, the Bacillus strain is a sporulation-negative mutant of a sporulation-positive mother strain selected from the group consisting of a Bacillus subtilis subsp. natto strain and a sporulation-negative Bacillus coagulans strain.

In a particular embodiment of the invention, the Bacillus strain is a sporulation-negative mutant of a sporulation-positive mother strain selected from the group consisting of DSM 32588, DSM 32589 and DSM 32606.

In a particular embodiment of the invention, the sporulation-negative Bacillus subtilis subsp. natto strain is selected from the group consisting of DSM 32892, DSM 32893, DSM 32894, DSM 32895 and mutants thereof. In the preceding sentence, the term “mutant” refers to a strain which is derived from one of the deposited strains disclosed herein by means of, e.g., genetic engineering, radiation and/or chemical treatment. It is preferred that the mutant is a functionally equivalent mutant, i.e. a mutant which has substantially the same or improved properties with respect to texture, shear stress, viscosity, viscoelasticity and/or gel stiffness as the deposited strain from which it was derived. Especially, the term “mutant” refers to strains obtained by subjecting a strain of the invention to any conventionally used mutagenization treatment including treatment with a chemical mutagen such as ethane methane sulphonate (EMS) or N-methyl-N′-nitro-N-nitroguanidine (NTG), UV light, or to a spontaneously occurring mutant. A mutant may have been subjected to several mutagenization treatments (a single treatment should be understood as one mutagenization step followed by a screening/selection step), but it is presently preferred that no more than 20, or no more than 10, or no more than 5, treatments (or screening/selection steps) are carried out. In a presently preferred mutant less than 1%, particularly less than 0.1%, less than 0.01%, more particularly less than 0.001%, and most particularly less than 0.0001% of the nucleotides in the bacterial genome have been replaced with another nucleotide, or deleted, compared to the mother strain. In a presently preferred mutant less than 50, particularly less than 30, more particularly less than 20, more particularly less than 10, and most particularly less than 5 the nucleotides in the bacterial genome have been replaced with another nucleotide, or deleted, compared to the mother strain.

The sporulation-negative strain of the invention may be obtained using any conventional method of producing a sporulation-negative mutant bacterium strain of a sporulation-positive bacterium mother strain.

A particular method for producing a sporulation-negative mutant bacterium strain of a sporulation-positive bacterium mother strain comprises the steps of
a) subjecting a culture of the mother strain to a mutagenesis treatment method,
b) growing the treated culture is on sporulation-inducing medium agar plates, and
c) selecting colonies of sporulation-negative mutants on the basis of visual inspection.

The said mutagenesis treatment method may be any conventionally used mutagenization treatment including treatment with a chemical mutagen, such as ethane methane sulphonate (EMS) or N-methyl-N′-nitro-N-nitroguanidine (NTG), and UV radiation. A mutant may have been subjected to several mutagenization treatments (a single treatment should be understood as one mutagenization step followed by a screening/selection step), but it is presently preferred that no more than 20, or no more than 10, or no more than 5, treatments (or screening/selection steps) are carried out. In a presently preferred mutant less than 1%, less than 0.1%, less than 0.01%, less than 0.001% or even less than 0.0001% of the nucleotides in the bacterial genome have been replaced with another nucleotide, or deleted, compared to the mother strain.

In a particular embodiment of the invention, the said mutagenesis treatment method is UV radiation.

The said sporulation-inducing medium of the agar plates may be any conventional sporulation-inducing medium, e.g. a commercial sporulation-inducing medium.

The said selection of colonies of sporulation-negative mutants on the basis of visual inspection is carried out so as to select colonies, which are paler and/or more translucent as compared to the colonies of sporulation-positive strains.

In the present invention the term “sporulation-negative strain” means a strain, which forms no spores when subjected to the following method:
i) inoculating 1% of a culture of the strain to be tested, grown over night in Veal Infusion Broth (VIB) at 37° C., 180 rpm, into 50 ml of a standard sporulation inducing medium contained in a 500 ml baffled shake flask,
ii) allowing the inoculated medium to grow overnight at 37° C. while subjecting it to shaking at 200 rpm, and
iii) testing for spores the next day.

The said standard sporulation-inducing medium may be any conventional sporulation-inducing medium, e.g. a commercial sporulation-inducing medium.

The said testing for spores may be carried out by the following method:
a) Heating aliquots of 1 ml of a culture of the strain to be tested at 80° C. for 10 min.,
b) Making 10-fold dilutions in peptone-salt water and spreading 100 pl of each dilution of the heat-treated sample on Blood Agar (BA) agar plates (only sporulation-positive strains can grow after the heat treatment),
c) Incubating the BA agar plates at 37° C., and
d) Checking for growth after 24 and 48 hours.

According to the invention, fermentation of the milk substrate with the lactic acid bacterium starter culture is performed in the presence of at least one Bacillus strain selected from the group consisting of a sporulation-negative Bacillus subtilis subsp. natto and a sporulation-negative Bacillus coagulans strain. Bacillus is a genus of Gram-positive, spore-forming bacteria which have attracted attention during the last years also in the food industry. Bacillus subtilis subsp. natto is known as a non-pathogenic bacterium which is utilized for manufacturing the traditional Japanese fermented soy food “natto”. Bacillus subtilis subsp. natto has received GRAS notification (“Generally Recognized as Safe”) by the FDA and can be purchased from different manufacturers. Bacillus coagulans has been used as a probiotic for its purported support of good digestive and immune health. It is used in some foods, including baked goods, dairy products, and grain products. Bacillus coagulans has also received GRAS notification by the FDA. Strains of Bacillus coagulans are commercially available from different manufacturers.

In a particular embodiment of the invention, the classification of a bacterium as a Bacillus subtilis subsp. natto strain according to the present invention is carried out by genome sequencing.

In a particular embodiment of the invention, the classification of a bacterium as a Bacillus coagulans strain according to the present invention is carried out by genome sequencing.

Process of the Invention

As used herein, “fermentation” means the conversion of carbohydrates or sugars into alcohols or acids through the action of a microorganism. Preferably, fermentation in the sense of the instant invention comprises the conversion of lactose to lactic acid. The fermentation of carbohydrates or sugars by lactic acid bacteria is particularly preferred.

In step (a) of the method of the invention, the milk substrate to be subjected to fermentation is provided. The term “milk substrate” refers to any raw and/or processed milk material that can be subjected to fermentation according to the method of the invention. As used herein, “milk” refers to the lacteal secretion obtained by milking a mammal, such as a cow, a sheep, a goat, a buffalo or a camel. Also included by the term “milk” are protein and/or fat solutions made of plant materials, in particular soy milk. In a preferred embodiment of the present invention, the milk used in the method of the present invention is cow milk.

Useful milk substrates include, but are not limited to, solutions/suspensions of milk or milk-like products comprising protein, such as whole milk or low fat milk, skim milk, buttermilk, reconstituted milk powder, condensed milk, dried milk, whey, whey permeate, lactose, mother liquid from crystallization of lactose, whey protein concentrate, or cream. Obviously, the milk substrate may originate from any mammal, e.g. being substantially pure mammalian milk, or reconstituted milk powder.

In step (b) of the method of the invention, the milk substrate selected for the fermentation process is fermented with a lactic acid bacterium starter culture. According to the present invention, a “lactic acid bacteria starter culture” or “lactic acid bacteria starter” is a composition which includes one or more lactic acid bacteria strains that shall be used for the fermentation. A starter culture is normally supplied either as a frozen or freeze-dried culture for bulk starter propagation or as so-called “Direct Vat Set” (DVS) cultures, i.e. a culture intended for the direct inoculation into a fermentation vessel or vat for the production of a dairy product, such as a fermented milk product. Prior to fermentation, the milk substrate may be subjected to homogenization or pasteurization. “Homogenization” refers to an intensive mixing to obtain a soluble suspension or emulsion. If homogenization is performed prior to fermentation, it may be performed so as to break up the milk fat globules into globules of smaller sizes to prevent the fat component from separating from the milk. This may be accomplished by forcing the milk at high pressure through small orifices. “Pasteurizing” refers to the treatment of the milk substrate to reduce or eliminate the presence of live organisms, such as microorganisms. Preferably, pasteurization is attained by maintaining the milk substrate at a specified temperature for a specified period of time. The specified temperature is usually attained by heating. The temperature and duration may be selected in order to kill or inactivate certain bacteria, such as harmful bacteria. A rapid cooling step may follow.

In the context of the present invention, the term “lactic acid bacterium” designates a gram-positive, microaerophilic or anaerobic bacterium which ferments sugars and thereby produces acids, including lactic acid, acetic acid and propionic acid. Normally, the acid which is predominantly produced acid is lactic acid. Lactic acid bacteria within the order “Lactobacillales” that have been found useful for industrial purposes include Lactococcus spp., Streptococcus spp., Lactobacillus spp., Leuconostoc spp., Pseudoleuconostoc spp., Pediococcus spp., Brevibacterium spp., Enterococcus spp. and Propionibacterium spp. Lactic acid bacteria also include the group of strictly anaerobic bifidobacteria, i.e. Bifidobacterium spp. They are frequently used as food cultures alone or in combination with other lactic acid bacteria.

While the milk substrate is to be fermented with the mesophilic lactic acid bacterium starter culture in the presence of the at least one Bacillus strain, it will not be necessary that the Bacillus strain is present during the complete fermentation time. It is sufficient that the at least one Bacillus strain is present for a substantial part of fermentation, e.g. for at least 50%, at least 60%, at least 70%, at least 80%, at least 90%, or at least 95% of the overall fermentation time. As used herein, “fermentation time” defines the time period between inoculation of the milk substrate and reaching the pre-determined pH.

For example, the milk substrate may be inoculated with the lactic acid bacterium starter culture, followed by incubation of the milk substrate for several hours, e.g. for 1-5 hours, such as for 2, 3 or 4 hours. Subsequently, the one or more Bacillus strains can be added to the milk substrate and fermentation can be continued for several hours until the desired pH has been reached. Conversely, the milk substrate may firstly be inoculated with the one or more Bacillus strains and incubated for several hours, preferably 1-5 hours, such as 2, 3 or 4 hours, followed by the addition of the mesophilic lactic acid bacterium starter culture. The successive inoculation of the milk substrate can be used as a means for adjusting the desired texture of gel stiffness.

In a particularly preferred embodiment, the bacteria from the lactic acid bacterium starter culture and the one or more Bacillus strains are present in the milk substrate for the complete fermentation time which means that the lactic acid bacterium starter culture and the one or more Bacillus strains are inoculated together into the milk substrate at the start of fermentation.

The starter culture may comprise as further components cryoprotectants and/or other conventional additives such as, colorants, yeast extract, sugars and vitamins.

In a particular embodiment, the lactic acid bacterium starter culture comprises at least one Lactococcus lactis strain. In the following this embodiment is referred to as a mesophilic starter culture.

In a particular embodiment, the lactic acid bacterium starter culture comprises at least one Streptococcus thermophilus strain and at least one Lactobacillus delbrueckii subsp. bulgaricus strain. In the following this embodiment is referred to as a thermophilic starter culture.

Process Using Mesophilic Starter Culture

The method of the invention aims at the production of a mesophilic fermented dairy product. A “mesophilic fermented dairy product” is a dairy product which has been prepared by fermentation with mesophilic microorganisms, and in particular mesophilic LAB. “Mesophilic” microorganisms have a growth optimum at moderate temperatures of between 15° C.-40° C. Typical LAB which are considered mesophilic include, but are not limited to, Lactococcus spp. and Leuconostoc spp. A “mesophilic fermentation” herein refers to fermentation at a temperature between 15° C.-35° C., preferably between 20° C.-35° C., and even more preferably between 25° C.-30° C. Typical dairy products which are considered “mesophilic fermented dairy products” include, but are not limited to, buttermilk, sour milk, cultured milk, smetana, sour cream and fresh cheese, such as quark, tvarog and cream cheese. In contrast, “thermophilic” microorganisms have a growth optimum at temperatures above 43° C. Thermophilic LAB that are used in the dairy industry include, amongst others, Streptococcus spp. and Lactobacillus spp. Accordingly, a “thermophilic fermentation” which is performed with thermophilic microorganisms normally uses a temperature above 35° C. The term “thermophilic dairy product” refers to dairy products prepared by fermentation with thermophilic microorganisms, and in particular thermophilic LAB. However, the thermophilic strains Streptococcus spp. are also used for producing some mesophilic fermented dairy products, e.g. in combination with the mesophilic strains Lactococcus spp., in which case a temperature of e.g. 25-35° C. is preferred, more preferably 30-35° C.

The fat content of the milk substrate depends on the specific substrate that is used. In a preferred embodiment of the invention, the method is used for preparing sour cream which means that the milk substrate used in the process is cream having a fat content of 6 to 45%, preferably 9% to 35%, more preferably 12% to 30%, more preferably 14% to 25% and most preferably 16% to 22%.

According to the invention, the mesophilic lactic acid bacteria starter culture includes at least one Lactococcus lactis strain. In one embodiment, the Lactococcus lactis strain is a Lactococcus lactis subsp. lactis strain. In another embodiment, the Lactococcus lactis strain is a Lactococcus lactis subsp. cremoris strain.

Apart from the at least one Lactococcus lactis strain, the mesophilic lactic acid bacterium starter culture may include additional mesophilic lactic acid bacteria, such as other strains of L. lactis subsp. lactis or L. Lactis subsp. cremoris. In a particular preferred embodiment, the mesophilic lactic acid bacterium starter culture includes one or more L. lactis subsp. lactis biovar. diacetylactis strains which produces flavor compounds. Alternatively, or in addition, the mesophilic starter culture may include one or more bacteria of the following genera: Leuconostoc, Pseudoleuconostoc, Pediococcus or Lactobacillus. Particularly preferred examples include Leuconostoc mesenteroides, Pseudoleuconostoc mesenteroides, Pediococcus pentosaceus, Lactobacillus casei and Lactobacillus paracasei. Particularly preferred examples include Leuconostoc mesenteroides subsp. cremoris, Pseudoleuconostoc mesenteroides subsp. cremoris, Pediococcus pentosaceus, Lactobacillus casei subsp. casei and Lactobacillus paracasei subsp. paracasei.

In a particularly preferred embodiment of the invention, the mesophilic lactic acid bacterium starter culture does not comprise a lactic acid bacterium that produces exopolysaccharides (EPS). In particular, the mesophilic lactic acid bacterium starter culture does not comprise a Streptococcus strain, such as a Streptococcus thermophilus strain.

Typically, the milk substrate, e.g. the cream for preparing sour cream, is inoculated with the mesophilic lactic acid bacterium starter culture so as to achieve a concentration of viable lactic acid bacteria in the milk substrate in the range of 10⁴ to 10¹² cfu (colony forming units) per ml of the milk substrate, preferably 10⁵ to 10¹¹ cfu per ml of the milk substrate, more preferably 10⁶ to 10¹⁰ cfu per ml of the milk substrate, and even more preferably 10⁷ to 10⁹ cfu per ml or 10⁷ to 10⁸ cfu per ml of the milk substrate. Accordingly, the concentration of viable lactic acid bacteria in the milk substrate, e.g. the cream for preparing sour cream, can be at least about 10⁴ cfu per ml of the milk substrate, at least about 10⁵ cfu per ml of the milk substrate, at least about 10⁶ cfu per ml of the milk substrate, at least about 10⁷ cfu per ml of the milk substrate, at least about 10⁸ cfu per ml of the milk substrate, at least about 10⁹ cfu per ml of the milk substrate, at least about 10¹⁰ cfu per ml of the milk substrate, or at least about 10¹¹ cfu per ml of the milk substrate.

Where the mesophilic lactic acid bacterium starter culture comprises a mixture of two or more different bacteria, it is preferred that the milk substrate, e.g. the cream for preparing sour cream, is inoculated to achieve a concentration of the Lactococcus lactis strains in the milk substrate of at least about 10³ cfu per ml of the milk substrate, at least about 10⁴ cfu per ml of the milk substrate, at least about 10⁵ cfu per ml of the milk substrate, at least about 10⁶ cfu per ml of the milk substrate, at least about 10⁷ cfu per ml of the milk substrate, or at least about 10⁸ cfu per ml of the milk substrate.

The Bacillus subtilis subsp. natto or a Bacillus coagulans strain will be inoculated into the milk substrate, e.g. the cream for preparing sour cream, such that after inoculation the concentration of the Bacillus strain will be comparable to that recited above in the context of the mesophilic lactic acid bacterium starter culture. This means that the milk substrate, e.g. the cream for preparing sour cream, is inoculated with the one or more Bacillus strains so as to achieve a concentration of viable Bacillus bacteria of the recited species in the milk substrate in the range of 10⁴ to 10¹² cfu per ml of the milk substrate, preferably 10⁵ to 10¹¹ cfu per ml of the milk substrate, more preferably 10⁶ to 10¹⁰ cfu per ml of the milk substrate, and even more preferably 10⁷ to 10⁹ cfu per ml or 10⁷ to 10⁸ cfu per ml of the milk substrate. Accordingly, the concentration of viable Bacillus bacteria of the recited species in the milk substrate can be at least about 10⁴ cfu per ml of the milk substrate, at least about 10⁵ cfu per ml of the milk substrate, at least about 10⁶ cfu per ml of the milk substrate, at least about 10⁷ cfu per ml of the milk substrate, at least about 10⁸ cfu per ml of the milk substrate, at least about 10⁹ cfu per ml of the milk substrate, at least about 10¹⁰ cfu per ml of the milk substrate, or at least about 10¹¹ cfu per ml of the milk substrate.

It is particularly preferred that the one or more Bacillus strains are added to the milk substrate in a concentration of 10⁷ to 10⁸ cfu/ml of the milk substrate. In another preferred embodiment, the Bacillus strain used in the method of the present invention produces significant amounts of vitamin K.

After inoculation with the mesophilic lactic acid bacterium starter culture and the one or more Bacillus strains, the milk substrate is incubated under conditions suitable for the propagation of the mesophilic lactic acid bacteria. This will preferably include a temperature of between 15° C.-35° C., more preferably between 20° C.-35° C., and even more preferably between 25° C.-35° C., such as between 26° C.-34° C. The specific temperature to be used during fermentation will mainly depend on the mesophilic fermented dairy product that shall be produced. For example, where the method is applied for the preparation of sour cream, the temperature during the fermentation will be 26-34° C., preferably 28-32° C.

Generally, the fermented dairy product which is produced by the method of the present invention can be any type of dairy product which usually is produced by means of mesophilic fermentation. In a preferred embodiment, however, the mesophilic fermented dairy product is selected from the group consisting of sour cream, sour milk, buttermilk, cultured milk, smetana, quark, tvarog, fresh cheese and cream cheese. In a preferred embodiment, the mesophilic fermented dairy product is sour cream.

The fermentation is carried out until the milk substrate reaches the desired pH which is normally between pH 4.0 and 5.0, and preferably between pH 4.5 and 4.8. Thus, the pH will be monitored during the fermentation process, and the fermentation will be stopped when the pre-determined pH is measured in the fermentation vessel. Depending on the concentration of the starter culture and the product to be manufactured, fermentation may take between 5-24 hours, preferably between 5-20 hours, more preferably between 5-16, more preferably between 5-14, more preferably between 6-12, more preferably between 7-11 and most preferably between 8-10 hours.

After fermentation, the fermented dairy product can be cooled and further processed. For example, depending on the type of fermented milk product the processing may include, e.g., the incubation of the product obtained from fermentation with enzymes, such as chymosin and pepsin. When the fermented milk product is a cheese, the processing may also include the cutting of the coagulum into cheese curd particles. The processing of the product may also include the packaging of the fermented milk product. A suitable package may be a bottle, a carton, or the like, having a volume of, e.g. 50 ml to 1000 ml.

The method of the invention has the particular advantage that when using a mesophilic lactic acid bacterium starter culture together with a Bacillus strain selected from the group consisting of a Bacillus subtilis subsp. natto strain and a Bacillus coagulans strain in the preparation of a mesophilic fermented dairy product, such as sour cream, the texture properties of the resulting dairy product, in particular viscosity, shear stress and gel stiffness, can be significantly improved.

Preferably, by using a method as defined herein, an increase in the shear stress, gel stiffness and/or gel firmness of the fermented dairy product of at least, 10%, at least 15%, at least 20%, at least 25%, at least 30%, at least 35%, at least 40%, at least 45%, at least 50%, at least 75%, at least 100° A), at least 150%, at least 200%, at least 250%, at least 300%, at least 350%, at least 400%, at least 450%, or at least 500% can be obtained relative to fermentation of the same milk substrate under identical conditions in the absence of any Bacillus strain.

In one preferred embodiment, the increase in shear stress of a fermented dairy product is at least 5, at least 10, at least 15, at least 20, at least 25, or at least 30 Pa relative to a corresponding fermented dairy product obtained by fermentation of the same milk substrate under identical conditions in the absence of any Bacillus strain.

In another preferred embodiment, the increase in the gel stiffness of a fermented dairy product is at least 25, at least 50, at least 100, at least 150, at least 200, at least 250 Pa, or at least 300 Pa, relative to a corresponding fermented dairy product obtained by fermentation of the same milk substrate under identical conditions in the absence of any Bacillus strain.

In yet another preferred embodiment, the increase in gel firmness of a fermented dairy product is at least 50 (g×sec), at least 75 (g×sec), at least 100 (g×sec), at least 125 (g×sec), at least 150 (g×sec), at least 175 (g×sec), or at least 200 (g×sec).

In a particular embodiment of the invention, shear stress is measured by the method defined in Example 2.

In a particular embodiment of the invention, gel stiffness is determined as Complex Modulus using the method defined in Example 2.

In a particularly preferred embodiment of the invention, the above-described method relates to the manufacturing of sour cream. Accordingly, in a particularly preferred embodiment a method for producing sour cream is provided, said method comprising:
(a) providing cream having a fat content of at least 6%,
(b) inoculating said cream with a mesophilic lactic acid bacterium starter culture comprising at least one Lactococcus lactis strain, and optionally additional mesophilic lactic acid bacteria,
(c) inoculating said cream with at least one Bacillus strain selected from the group consisting of a Bacillus subtilis subsp. natto strain and a Bacillus coagulans strain,
(d) fermenting said cream until the pH reaches 4.0 to 5.0, more preferably 4.5 to 4.6,
(e) obtaining the sour cream.

In a first step of the above method, cream is provided as a milk substrate. The cream used for the process of manufacturing sour cream is preferably obtained from cow milk. The fat content of the cream will be at least 6% which is the usual fat content for cream that is used in the production of sour cream. Typically, the fat content is standardized prior to fermentation to comply with food regulations. During standardization, dry ingredients may be added to the cream such as whey or caseins. If stabilizers are to be added, they may also be added at this stage of the preparation process. Suitable stabilizers include, for example, polysaccharides, starch and gelatin.

Subsequently, the cream is preferably subjected to homogenization in order to break down larger fat globules into smaller globules, thereby providing an even suspension in preventing the separation of the whey. Homogenization of the cream can be carried out in a standard homogenizer which is routinely used in the dairy industry. Homogenization conditions may comprise a pressure of 100 to 200 bar, preferably 130 to 150 bars and a temperature of between 50 and 80° C., preferably between 65° C. and 75° C. In a particular embodiment, homogenization is carried out in two steps at 150-200 bar and 65° C. to 75° C. in a first step and at 30-60 bar and 65° C. to 75° C. in a second step.

After homogenization, the cream may undergo pasteurization to kill potentially harmful bacteria. Preferably, pasteurization is carried out as a high temperature short time (HTST) pasteurization, which normally means that the cream is heated to 80 to 90° C. and incubated at that temperature for about 2 to 10 minutes, in particular 2-5 minutes. After pasteurization, the cream is cooled down to the selected fermentation temperature for inoculation of the mesophilic lactic acid bacterium starter culture.

The cream is then inoculated with a mesophilic lactic acid bacterium starter culture as defined above which comprises at least one Lactococcus lactis strain, and optionally additional mesophilic lactic acid bacteria. Normally the cream is inoculated with 0.01-0.02% starter culture. The inoculated cream is then normally incubated for about 12 to 18 hours until a pH of 4.5 to 4.6 is reached. Once the pre-determined pH is reached, the fermented sour cream product is cooled and packaged.

Composition Comprising a Mesophilic Starter Culture

A particular embodiment of the invention relates to a composition for producing a mesophilic fermented dairy product, comprising
(a) a lactic acid bacterium starter culture comprising at least one Lactococcus lactis strain, and
(b) a Bacillus strain selected from the group consisting of a sporulation-negative Bacillus subtilis subsp. natto strain and a sporulation-negative Bacillus coagulans strain, and wherein a sporulation-negative strain is a strain, which forms no spores when subjected to the following method:

i) inoculating 1% of a culture of the strain to be tested, grown over night in Veal Infusion Broth (VIB) at 37° C., 180 rpm, into 50 ml of a standard sporulation inducing medium contained in a 500 ml baffled shake flask,

ii) allowing the inoculated medium to grow overnight at 37° C. while subjecting it to shaking at 200 rpm, and

iii) testing for spores the next day.

The composition can be formulated for being suitable for direct inoculation of a milk substrate or another culture medium prior to fermentation.

Fermented Dairy Product Comprising a Mesophilic Starter Culture

A particular embodiment of the invention relates to a mesophilic fermented dairy product obtainable by the method of the invention.

A particular embodiment of the invention relates to a mesophilic fermented dairy product, comprising
(a) a lactic acid bacterium starter culture comprising a Lactococcus lactis strain, and
(b) a Bacillus strain selected from the group consisting of a sporulation-negative Bacillus subtilis subsp. natto strain and a sporulation-negative Bacillus coagulans strain, and wherein a sporulation-negative strain is a strain, which forms no spores when subjected to the following method:

i) inoculating 1% of a culture of the strain to be tested, grown over night in Veal Infusion Broth (VIB) at 37° C., 180 rpm, into 50 ml of a standard sporulation inducing medium contained in a 500 ml baffled shake flask,

ii) allowing the inoculated medium to grow overnight at 37° C. while subjecting it to shaking at 200 rpm, and

iii) testing for spores the next day.

The Lactococcus lactis strain which is present in the mesophilic composition according to the invention or in the mesophilic fermented dairy product according to the invention is preferably selected from the group consisting of Lactococcus lactis subsp. lactis or Lactococcus lactis subsp. cremoris.

Apart from the at least one Lactococcus lactis strain, the composition according to the invention or the mesophilic fermented dairy product according to the invention may include additional mesophilic lactic acid bacteria, such as other strains of L. lactis subsp. lactis, L. lactis subsp. lactis biovar. diacetylactis, or L. Lactis subsp. cremoris. Alternatively, the composition according to the invention or the mesophilic fermented dairy product according to the invention may include mesophilic bacteria of the genus Leuconostoc, Pseudoleuconostoc, Pediococcus or Lactobacillus. Particularly preferred examples include Leuconostoc mesenteroides, Pseudoleuconostoc mesenteroides, Pediococcus pentosaceus, Lactobacillus casei and Lactobacillus paracasei. Particularly preferred examples include Leuconostoc mesenteroides subsp. cremoris, Pseudoleuconostoc mesenteroides subsp. cremoris, Pediococcus pentosaceus, Lactobacillus casei subsp. casei and Lactobacillus paracasei subsp. paracasei.

The mesophilic fermented dairy product according to the invention preferably is selected from the group consisting of sour cream, sour milk, buttermilk, cultured milk, smetana, quark, tvarog, fresh cheese and cream cheese, and more preferably is sour cream.

Process Using Thermophilic Starter Culture

A “thermophilic” microorganism has a growth optimum at temperatures above 43° C. Thermophilic LAB that are used in the dairy industry include, amongst others, Streptococcus spp. and Lactobacillus spp. Accordingly, a “thermophilic fermentation” which is performed with thermophilic microorganisms normally uses a temperature above 35° C. The term “thermophilic dairy product” refers to dairy products prepared by fermentation with thermophilic microorganisms, and in particular thermophilic LAB.

Most conventional starter cultures used for producing various types of fermented milk products are suitable for use in the process of the invention. Preferred starter cultures are those, which produce fermented milk products with high texture and/or texture. Also, it is preferred that the fermented milk product produced is resistant to subsequent heat treatment.

In a preferred embodiment of the invention, the starter culture comprises one or more Lactic Acid Bacteria (LAB) strains selected from the group consisting of lactic acid bacteria strains from the order “Lactobacillales”. Preferably, the starter culture comprises one or more Lactic Acid Bacteria (LAB) strains selected from the group consisting of Lactococcus spp., Streptococcus spp., Lactobacillus spp., Leuconostoc spp., Pseudoleuconostoc spp., Pediococcus spp., Brevibacterium spp., Enterococcus spp. and Propionibacterium spp.

Typically, the milk substrate is inoculated with the mesophilic lactic acid bacterium starter culture so as to achieve a concentration of viable lactic acid bacteria in the milk substrate in the range of 10⁴ to 10¹² cfu (colony forming units) per ml of the milk substrate, preferably 10⁵ to 10¹¹ cfu per ml of the milk substrate, more preferably 10⁶ to 10¹⁰ cfu per ml of the milk substrate, and even more preferably 10⁷ to 10⁹ cfu per ml or 10⁷ to 10⁸ cfu per ml of the milk substrate. Accordingly, the concentration of viable lactic acid bacteria in the milk substrate, e.g. the cream for preparing sour cream, can be at least about 10⁴ cfu per ml of the milk substrate, at least about 10⁵ cfu per ml of the milk substrate, at least about 10⁶ cfu per ml of the milk substrate, at least about 10⁷ cfu per ml of the milk substrate, at least about 10⁸ cfu per ml of the milk substrate, at least about 10⁹ cfu per ml of the milk substrate, at least about 10¹⁰ cfu per ml of the milk substrate, or at least about 10¹¹ cfu per ml of the milk substrate.

Where the thermophilic lactic acid bacterium starter culture comprises a mixture of two or more different bacteria, it is preferred that the milk substrate is inoculated to achieve a concentration of the Streptococcus thermophilus strain in the milk substrate of at least about 10³ cfu per ml of the milk substrate, at least about 10⁴ cfu per ml of the milk substrate, at least about 10⁵ cfu per ml of the milk substrate, at least about 10⁶ cfu per ml of the milk substrate, at least about 10² cfu per ml of the milk substrate, or at least about 10⁸ cfu per ml of the milk substrate.

The Bacillus subtilis subsp. natto or a Bacillus coagulans strain will be inoculated into the milk substrate such that after inoculation the concentration of the Bacillus strain will be comparable to that recited above in the context of the thermophilic lactic acid bacterium starter culture. This means that the milk substrate is inoculated with the one or more Bacillus strains so as to achieve a concentration of viable Bacillus bacteria of the recited species in the milk substrate in the range of 10⁴ to 10¹² cfu per ml of the milk substrate, preferably 10⁵ to 10¹¹ cfu per ml of the milk substrate, more preferably 10⁶ to 10¹⁰ cfu per ml of the milk substrate, and even more preferably 10⁷ to 10⁹ cfu per ml or 10⁷ to 10⁸ cfu per ml of the milk substrate. Accordingly, the concentration of viable Bacillus bacteria of the recited species in the milk substrate can be at least about 10⁴ cfu per ml of the milk substrate, at least about 10⁵ cfu per ml of the milk substrate, at least about 10⁶ cfu per ml of the milk substrate, at least about 10⁷ cfu per ml of the milk substrate, at least about 10⁸ cfu per ml of the milk substrate, at least about 10⁹ cfu per ml of the milk substrate, at least about 10¹⁰ cfu per ml of the milk substrate, or at least about 10¹¹ cfu per ml of the milk substrate.

It is particularly preferred that the one or more Bacillus strains are added to the milk substrate in a concentration of 10⁷ to 10⁸ cfu/ml of the milk substrate. In another preferred embodiment, the Bacillus strain used in the method of the present invention produces significant amounts of vitamin K.

In the process of the invention, it is preferred that the starter culture has an acidification capacity so that the fermented milk product reaches a pH of 4.3 in less than 12 hours, preferably less than 10 hours, more preferably less than 9 hours, more preferably less than 8 hours, and most preferably less than 7 hours.

In a preferred embodiment of the process of the invention the target pH is from 3.80 to 4.39, preferably from 3.80 to 4.38, more preferably from 3.80 to 4.37, more preferably from 3.80 to 4.36, more preferably from 3.80 to 4.35, more preferably from 3.80 to 4.34, more preferably from 3.80 to 4.33, more preferably from 3.80 to 4.32, more preferably from 3.80 to 4.31, more preferably from 3.80 to 4.30, more preferably from 3.90 to 4.30, and most preferably from 4.00 to 4.30.

In a preferred embodiment of the invention, the milk substrate used for the fermentation with the starter culture has a protein content of between 1% by weight (w/w) and 8.0% by weight (w/w), preferably between 1.2% by weight (w/w) and 7.0% by weight (w/w), more preferably between 1.4% by weight (w/w) and 6.0% by weight (w/w) preferably between 1.6% by weight (w/w) and 5.0% by weight (w/w), preferably between 1.8% by weight (w/w) and 4.5% by weight (w/w), and most preferably between 2.0% by weight (w/w) and 4.0% by weight (w/w).

In a preferred embodiment of the invention, the milk substrate used for the fermentation with the starter culture has a fat content of between 1% by weight (w/w) and 8.0% by weight (w/w), preferably between 1.2% by weight (w/w) and 7.0% by weight (w/w), more preferably between 1.4% by weight (w/w) and 6.0% by weight (w/w) preferably between 1.6% by weight (w/w) and 5.0% by weight (w/w), preferably between 1.8% by weight (w/w) and 4.5% by weight (w/w), and most preferably between 2.0% by weight (w/w) and 4.0% by weight (w/w).

In a preferred embodiment the concentration of Streptococcus thermophilus cells inoculated is from 10⁴ to 10⁹ CFU Streptococcus thermophilus cells per ml of milk substrate, such as from 10⁴ CFU to 10⁸ CFU Streptococcus thermophilus cells per ml of milk substrate.

Preferably, by using a method as defined herein, an increase in the shear stress, gel stiffness and/or gel firmness of the fermented dairy product of at least, 10%, at least 15%, at least 20%, at least 25%, at least 30%, at least 35%, at least 40%, at least 45%, at least 50%, at least 75%, at least 100%, at least 150%, at least 200%, at least 250%, at least 300%, at least 350%, at least 400%, at least 450%, or at least 500% can be obtained relative to fermentation of the same milk substrate under identical conditions in the absence of any Bacillus strain.

In one preferred embodiment, the increase in shear stress of a fermented dairy product is at least 5, at least 10, at least 15, at least 20, at least 25, or at least 30 Pa relative to a corresponding fermented dairy product obtained by fermentation of the same milk substrate under identical conditions in the absence of any Bacillus strain.

In another preferred embodiment, the increase in the gel stiffness of a fermented dairy product is at least 25, at least 50, at least 100, at least 150, at least 200, at least 250 Pa, or at least 300 Pa, relative to a corresponding fermented dairy product obtained by fermentation of the same milk substrate under identical conditions in the absence of any Bacillus strain.

In yet another preferred embodiment, the increase in gel firmness of a fermented dairy product is at least 50 (g×sec), at least 75 (g×sec), at least 100 (g×sec), at least 125 (g×sec), at least 150 (g×sec), at least 175 (g×sec), or at least 200 (g×sec).

In a particular embodiment of the invention, shear stress is measured by the method defined in Example 2.

In a particular embodiment of the invention, gel stiffness is determined as Complex Modulus using the method defined in Example 2.

Composition Comprising a Thermophilic Starter Culture

A composition for producing a thermophilic fermented dairy product, comprising
(a) a lactic acid bacterium starter culture comprising at least one Streptococcus thermophilus strain and at least one Lactobacillus delbrueckii subsp. bulgaricus strain, and
(b) a Bacillus strain selected from the group consisting of a sporulation-negative Bacillus subtilis subsp. natto strain and a sporulation-negative Bacillus coagulans strain, and wherein a sporulation-negative strain is a strain, which forms no spores when subjected to the following method:

i) inoculating 1% of a culture of the strain to be tested, grown over night in Veal Infusion Broth (VIB) at 37° C., 180 rpm, into 50 ml of a standard sporulation inducing medium contained in a 500 ml baffled shake flask,

ii) allowing the inoculated medium to grow overnight at 37° C. while subjecting it to shaking at 200 rpm, and

iii) testing for spores the next day.

Fermented Dairy Product Comprising a Thermophilic Starter Culture

The thermophilic fermented milk product according to the present invention refers to fermented milk products prepared by thermophilic fermentation of a thermophilic starter culture and it is selected from the group including set-yoghurt, stirred-yoghurt and drinking yoghurt, e.g. Yakult.

A particular embodiment of the invention relates to a thermophilic fermented dairy product, comprising
(a) a lactic acid bacterium starter culture comprising a at least one Streptococcus thermophilus strain and at least one Lactobacillus delbrueckii subsp. bulgaricus strain, and
(b) a Bacillus strain selected from the group consisting of a sporulation-negative Bacillus subtilis subsp. natto strain and a sporulation-negative Bacillus coagulans strain, and wherein a sporulation-negative strain is a strain, which forms no spores when subjected to the following method:

i) inoculating 1% of a culture of the strain to be tested, grown over night in Veal Infusion Broth (VIB) at 37° C., 180 rpm, into 50 ml of a standard sporulation inducing medium contained in a 500 ml baffled shake flask,

ii) allowing the inoculated medium to grow overnight at 37° C. while subjecting it to shaking at 200 rpm, and

iii) testing for spores the next day.

### EXAMPLES

### Example 1

### Obtaining Sporulation-Negative Mutants of Bacillus subtilis subsp. natto DSM 32589

Bacillus subtilis subsp. natto DSM 32589 was used as mother strain. Sporulation-negative mutants of the mother strain were obtained by UV mutagenesis followed by selection of sporulation-negative phenotype and stability testing.

Materials:

Difco Sporulation Medium (DSM) (Per Liter):

| | |
|---|---|
| Bacto Nutrient broth (Difco 234000) | 8 g |
| 10% (w/v) KCl | 10 ml |
| 1.2% (w/v) MgSO4 7H2O | 10 ml |
| Agar | 15 g |
| 1M NaOH | ~1.5 ml (pH to 7.6) |

Adjust volume to 1 liter with double-distilled H2O. Adjust pH to 7.6. Autoclave and allow to cool to 50° C. if to be used immediately. Just prior to use, add the following sterile filtrated solutions:

| | |
|---|---|
| 1M Ca(NO3)2 | 1 ml (23.62 g/100 ml, depending of the MW) |
| 0.01M MnCl2 | 1 ml (0.31 g/100 ml with 4H2O) |
| 1 mM FeSO4 | 1 ml (0.051 g/100 ml with 7H2O) |

Selection:

4×4 ml of an undiluted culture of the mother strain were placed in open Petri dishes and put into a UV-crosslinker (Amersham Life Science) and exposed to the highest effect possible, 70 mJ/cm2 for four different time schedules: 2 min., 5 min., 2×5 min. and 4×5 min. Petri dishes were swirled every 5 minutes to avoid excessive heating of the culture.

Immediately after UV radiation, 2×1 ml of cells were inoculated into 2×5 ml Veal Infusion Broth (VIB), Difco 234420. Viability of cells after UV radiation were determined by plating 10-fold dilutions in duplicate on Blood Agar (BA) plates. Both plates and tubes were incubated overnight at 37° C. in the dark (175 rpm for tubes).

TABLE 1

| | | CFU/ml | |
|---|---|---|---|
| Sample | Overnight OD | T = 0: 3.4 × 10exp07 | % survivors |
| 2 min. | 4.886 | 6.2 × 10exp06 | 18.2 |
| 5 min. | 4.642 | 4.3 × 10exp06 | 12.6 |
| 2 × 5 min. | 3.900 | 2.4 × 10exp06 | 7.1 |
| 4 × 5 min. | 4.507 | 6.9 × 10exp04 | 0.2 |

Each of the four samples were deposited in in-house culture collection.

The two UV mutant pool samples 2×5 min. and 4×5 min. were spread on sporulation inducing agar (Difco Sporulation Medium, DSM) and incubated at 37° C. for 3 days. The sporulation-negative phenotype was identified as lysed colonies or colonies looking paler/more translucent than the sporulation-positive colonies. Four sporulation-negative colonies were obtained from the 2×5 min. sample and 23 sporulation-negative colonies were obtained from the 4×5 min. sample. The selected colonies were spread on DSM agar for purity and checked for spores by microscopy.

Five colonies from the 4×5 min. sample were selected and later deposited as DSM 32892, DSM 32893, DSM 32894, DSM 32895 and DSM 33182.

Stability Testing:

All strains obtained were subjected to stability testing.

Stability testing was performed in 96 deepwell microplates, square welled.

Four daily transfers to fresh sporulation inducing broth, 500 μl per well (Difco Sporulation Medium, DSM), incubation at 37° C., shaking 200 rpm. Testing for spores was done after the last transfer.

In addition, a run over the weekend (3 days growth) was made with a DSM medium, 37° C., 200 rpm.

Testing for Spores:

Two aliquots of 75 μl of each strain were heated at 90° C. for 15 min. and at 80° C. for 10 min. in a PCR machine. For each strain and for each heat treatment 10 μl was spotted on BA agar and checked for growth after 24 and 48 hours at 37° C.—only sporulation-positive strains will grow. All strains were consistently sporulation-negative, also when checked by microscopy.

Sequencing:

Two of the strains were genome sequenced, DSM 32892 and DSM 32893.

Results for DSM 32892: 2 mutations in genes unknown to be sporulation related.

Results for DSM 32893: 1 mutation in the spo0F gene, known to be essential for sporulation. 1 mutation in a region between 2 genes unknown to be sporulation related.

### Example 2:

### Sporulation-Negative Bacillus subtilis subsp. natto Reduces Acidification Time and Improves Rheology During Sour Cream Preparation

15 of the sporulation-negative Bacillus subtilis subsp. natto strains produced in Example 1 were tested for their texturizing properties in low fat sour cream in co-culture with a mesophilic starter culture (in the following referred to as MO-1). Four strains originated from the 2×5 min. sample of Example 1 and 11 strains originated from the 4×5 min. sample of Example 1. All five strains deposited as DSM 32892, DSM 32893, DSM 32894, DSM 32895 and DSM 33182 were tested.

Strains

Mesophilic starter (MO-1): Lactococcal starter culture comprising a number of Lactococcos lactis subsp. lactis strains and a number of Lactococcus lactis subsp. cremoris strains.

Milk Substrate

TABLE 2

| | Composition of final | |
|---|---|---|
| Ingredient | base | Procedure |
| Cream (9% & 38%), | 9% fat | Homogenization: |
| Milk protein concen- | 5% protein | 200/80 bar 70° C. |
| trate 85% | | Heat treatment: |
| | | 85° C., 3 minutes |

Fermentation

The fermentation was carried out in 200 ml low fat sour cream milk base at a temperature of 30° C. until a target pH of 4.55 was reached. The pH was measured by pH electrodes. The MO-1 culture was added to the milk substrate in a concentration of 0.01%. The Bacillus strains were added to the milk substrate to reach a final cell count of 10exp08 cells per ml.

Measurements

Complex Modulus and Shear Stress

Two days after production, the fermented milk product was brought to 13° C. and manually stirred gently by means of a spoon (5 times) until homogeneity of the sample. The rheological properties of the sample were assessed on a rheometer (Anton Paar Physica Rheometer with ASC, Automatic Sample Changer, Anton Paar® GmbH, Austria) by using a bob-cup. The rheometer was set to a constant temperature of 13° C. during the time of measurement. Settings were as follows:

Holding time (to rebuild to somewhat original structure)

5 minutes without any physical stress (oscillation or rotation) applied to the sample.

Oscillation step (to measure the elastic and viscous modulus, G′ and G″, respectively, therefore calculating the complex modulus G*)
Constant strain=0.3%, frequency (f)=[0.5 . . . 8] Hz
6 measuring points over 60 s (one every 10 s)

Rotation step (to measure shear stress at 300 1/s)

Two steps were designed:
1) Shear rate=[0.3-300] 1/s and 2) Shear rate=[275-0.3] 1/s.

Each step contained 21 measuring points over 210 s (on every 10 s).

The shear stress at the peak point of the flow curves was chosen for further analysis.

The Complex Modulus G* is a parameter, which expresses Gel Stiffness.

Results—Acidification

TABLE 3

| | Time to pH 4.55 | Difference in Time to pH 4.55 |
|---|---|---|
| | (hours minutes) | compared to MO-1 alone |
| MO-1 | 11 h | |
| DSM 32589 (Mother | 9 h | −2 h |
| strain in Exam-ple 1) | | |
| DSM 32982 | 9 h | −2 h |
| DSM 32983 | 8 h 30 min | −2 h 30 min |
| Spo(−) mutant 1 | 8 h 30 min | −2 h 30 min |
| Spo(−) mutant 2 | 8 h 30 min | −2 h 30 min |
| Spo(−) mutant 3 | 10 h | −1 h |
| Spo(−) mutant 5 | 9 h | −2 h |
| Spo(−) mutant 9 | 8 h 30 min | −2 h 30 min |
| Spo(−) mutant 13 | 8 h 30 min | −2 h 30 min |
| DSM 32894 | 9 h | −2 h |
| DSM 33182 | 8 h | −3 h |
| Spo(−) mutant 20 | 8 h | −3 h |
| Spo(−) mutant 22 | 8 h | −3 h |
| Spo(−) mutant 24 | 8 h | −3 h |
| Spo(−) mutant 26 | 8 h | −3 h |
| DSM 32895 | 8 h | −3 h |

Spo(−): sporulation-negative

As will appear from Table 3, the acidification time is significantly reduced for all 15 culture blends comprising a sporulation-negative mutant of the sporulation-positive mother strain DSM 32589 as compared to the starter culture with no Bacillus strain. Furthermore, the acidification time is significantly lower for 14 of the 15 culture blends comprising a sporulation-negative mutant of the sporulation-positive mother strain DSM 32589 as compared to the starter culture with the sporulation-positive mother strain DSM 32589.

Results—Texturizing Capacity

TABLE 4

| | Shear stress | Shear stress | Complex |
|---|---|---|---|
| | at 30.2 | at 30o Hz | modulus G* |
| | Hz (Pa) | (Pa) | at 2.64 Hz (Pa) |
| MO-1 | 90.00 | 104.00 | 699 |
| DSM 32589 (Mother | 95.6 | 111.00 | 883 |
| strain in Example 1) | | | |
| DSM 32892 | 95.40 | 108.00 | 801 |
| DSM 32893 | 93.30 | 106.00 | 775 |
| Spo(−) mutant 1 | 95.50 | 106.00 | 788 |
| Spo(−) mutant 2 | 95.90 | 106.00 | 805 |
| Spo(−) mutant 3 | 103.00 | 113.00 | 878 |
| Spo(−) mutant 5 | 106.00 | 113.00 | 937 |
| Spo(−) mutant 9 | 96.50 | 104.00 | 798 |
| Spo(−) mutant 13 | 98.300 | 107.00 | 819 |
| DSM 32894 | 102.00 | 109.00 | 908 |
| DSM 33182 | 98.90 | 106.00 | 813 |
| Spo(−) mutant 20 | 107.00 | 110.00 | 897 |
| Spo(−) mutant 22 | 104.00 | 109.00 | 884 |
| Spo(−) mutant 24 | 101.00 | 106.00 | 831 |
| Spo(−) mutant 26 | 105.00 | 108.00 | 903 |
| DSM 32895 | 107.00 | 111.00 | 946 |

Spo(−): sporulation-negative

As will appear from Table 4, the shear stress and Complex Modulus (Gel Stiffness) were significantly increased for all 15 culture blends comprising a sporulation-negative mutant of the sporulation-positive mother strain DSM 32589 as compared to the starter culture with no Bacillus strain. Furthermore, for shear stress at 30.2 Hz, the shear stress is significantly higher for 13 of the 15 culture blends comprising a sporulation-negative mutant of the sporulation-positive mother strain DSM 32589 as compared to the starter culture with the sporulation-positive mother strain DSM 32589. Furthermore, for shear stress at 300 Hz, the shear stress for all 15 culture blends comprising a sporulation-negative mutant of the sporulation-positive mother strain DSM 32589 is at the same level as compared to the starter culture with the sporulation-positive mother strain DSM 32589. Furthermore, the Complex Modulus is significantly higher for 6 of the 15 culture blends comprising a sporulation-negative mutant of the sporulation-positive mother strain DSM 32589 as compared to the starter culture with the sporulation-positive mother strain DSM 32589.

Overall, the texturizing capacity of the 15 sporulation-negative mutants of the sporulation-positive mother strain DSM 32589 is at the same level as the high level of the sporulation-positive mother strain DSM 32589.

### Example 3

### Sporulation-Negative Bacillus subtilis subsp. natto Reduces Acidification Time and Improves Rheology During Yogurt Preparation

15 of the sporulation-negative Bacillus subtilis subsp. natto strains produced in Example 1 (same 15 strains tested in Example 2) were tested for their texturizing properties in a yogurt milk substrate in co-culture with a commercial thermophilic starter culture Yoflex Premium 1.0 (in the following referred to as Premium).

Strains

Thermophilic yogurt starter culture: Commercial Yoflex Premium 1.0 containing a number of Streptococcus thermophilus and Lactobacillus delbrueckii subsp. bulgaricus strains.

Milk Substrate

TABLE 5

| Ingredient | Amount | Composition | Procedure |
|---|---|---|---|
| Skim Milk Powder | 9.5% | 0.1% fat | Heat treatment 98° C., |
| and water | | 2.5% protein | 30 minutes |

Fermentation

The fermentation was carried out at a temperature of 43° C. until a target pH of 4.55 was reached. The pH was measured by pH electrodes. The Bacillus strains were added to the milk substrate to reach a final cell count of 10exp08 cells per ml.

Measurements

Complex Modulus and Shear Stress

Complex Modulus and shear stress were measured in the same manner as in Example 2.

Results—Acidification

TABLE 6

| | Time to pH 4.55 | Difference in Time to pH 4.55 |
|---|---|---|
| | (hours min) | compared to Premium alone |
| Premium | 6 h 10 min | |
| DSM 32589 (Mother | 5 h 05 min | −1 h 05 min |
| strain in Example 1) | | |
| DSM 32892 | 5 h 30 min | −40 min |
| DSM 32893 | 5 h 30 min | −40 min |
| Spo(−) mutant 1 | 6 h 00 min | −10 min |
| Spo(−) mutant 2 | 6 h 00 min | −10 min |
| Spo(−) mutant 3 | 5 h 30 min | −40 min |
| Spo(−) mutant 5 | 6 h 20 min | +10 min |
| Spo(−) mutant 9 | 6 h 00 min | −10 min |
| Spo(−) mutant 13 | 5 h 45 min | −25 min |
| DSM 32894 | 5 h 45 min | −25 min |
| DSM 33182 | 5 h 20 min | −50 min |
| Spo(−) mutant 20 | 6 h 15 min | +05 min |
| Spo(−) mutant 22 | 5 h 30 min | −40 min |
| Spo(−) mutant 24 | 5 h 40 min | −30 min |
| Spo(−) mutant 26 | 5 h 40 min | −30 min |
| DSM 32895 | 5 h 40 min | −30 min |

Spo(−): sporulation-negative

As will appear from Table 6, the acidification time is significantly reduced for all 13 of 15 culture blends comprising a sporulation-negative mutant of the sporulation-positive mother strain DSM 32589 as compared to the starter culture with no Bacillus strain. Furthermore, the acidification time is slightly higher for the 15 culture blends comprising a sporulation-negative mutant of the sporulation-positive mother strain DSM 32589 as compared to the starter culture with the sporulation-positive mother strain DSM 32589.

Results—Texturizing Capacity

TABLE 7

| | Shear stress | Shear stress | Complex |
|---|---|---|---|
| | at 30.2 | at 300 Hz | modulus G* |
| | Hz (Pa) | (Pa) | at 2.64 Hz (Pa) |
| Premium | 23.80 | 39.20 | 117 |
| DSM 32589 (Mother | 26.30 | 47.90 | 118 |
| strain in Example 1) | | | |
| DSM 32892 | 29.80 | 54.20 | 120 |
| DSM 32893 | 27.20 | 50.40 | 112 |
| Spo(−) mutant 1 | 30.60 | 53.10 | 129 |
| Spo(−) mutant 2 | 28.90 | 51.40 | 128 |
| Spo(−) mutant 3 | 29.70 | 51.20 | 123 |
| Spo(−) mutant 5 | 30.00 | 50.60 | 134 |
| Spo(−) mutant 9 | 29.40 | 51.20 | 124 |
| Spo(−) mutant 13 | 28.80 | 51.50 | 124 |
| DSM 32894 | 26.30 | 50.70 | 112 |
| DSM 33182 | 26.90 | 51.70 | 112 |
| Spo(−) mutant 20 | 29.10 | 49.90 | 128 |
| Spo(−) mutant 22 | 27.10 | 50.70 | 116 |
| Spo(−) mutant 24 | 26.50 | 49.30 | 111 |
| Spo(−) mutant 26 | 27.50 | 51.10 | 120 |
| DSM 32895 | 29.10 | 52.40 | 125 |

Spo(−): sporulation−negative

As will appear from Table 7, the shear stress at both 30.2 Hz and 300 Hz were significantly increased for all 15 culture blends comprising a sporulation-negative mutant of the sporulation-positive mother strain DSM 32589 as compared to the starter culture with no Bacillus strain. Also, the Complex Modulus were increased for all 11 culture blends comprising a sporulation-negative mutant of the sporulation-positive mother strain DSM 32589 as compared to the starter culture with no Bacillus strain.

Furthermore, the shear stress at both 30.2 Hz and 300 Hz were significantly increased for all 15 culture blends comprising a sporulation-negative mutant of the sporulation-positive mother strain DSM 32589 as compared to the starter culture with the sporulation-positive mother strain DSM 32589. Furthermore, the Complex Modulus is significantly higher for 10 of the 15 culture blends comprising a sporulation-negative mutant of the sporulation-positive mother strain DSM 32589 as compared to the starter culture with the sporulation-positive mother strain DSM 32589.

Overall, the texturizing capacity of the 15 sporulation-negative mutants of the sporulation-positive mother strain DSM 32589 is at an even higher level than the high level of the sporulation-positive mother strain DSM 32589.

### Example 4

### Sporulation-Negative Bacillus subtilis subsp. natto Strain DSM 33181 Reduces Acidification Time and Improves Rheology During Yogurt Preparation

The strain deposited as DSM 33181 is one of the 23+4 strains produced in Example 1. The strain was tested with respect to acidification time and shear stress 1) in a co-culture with mesophilic starter culture for producing sour cream and 2) in a co-culture with a yogurt starter culture for producing yogurt. For comparison, the corresponding starter cultures without any Bacillus strain and with the sporulation-positive Bacillus mother strain were used.

For the production of sour cream, the mesophilic starter culture, the milk substrate, the fermentation procedure and the measurements are the same as described in Example 2. For the production of yogurt, the yogurt starter culture, the milk substrate, the fermentation procedure and the measurements are the same as described in Example 3.

Results—Acidification

TABLE 8

| | Difference in Time to pH 4.55 com- |
|---|---|
| | pared to MO-1 or Premium alone |
| | (hours) |
| MO-1 | |
| MO-1 + DSM 32589 | −1.26 h |
| (Mother strain in Example 1) | |
| MO-1 + DSM 33181 | −0.50 h |
| Premium | |
| Premium + DSM 32589 | −1.84 h |
| (Mother strain in Example 1) | |
| Premium + DSM 33181 | −2.34 h |

As will appear from Table 8, the acidification time is significantly reduced for both the culture blend comprising DSM 33181 and for the culture blend comprising the sporulation-positive mother strain DSM 32589 as compared to the starter culture with no Bacillus strain.

Results—Texturizing Capacity

TABLE 9

| | Shear stress at | Shear stress at |
|---|---|---|
| | 75.2 Hz (Pa) | 300 Hz (Pa) |
| MO-1 | 118.0 | 104.0 |
| MO-1 + DSM 32589 | 131.0 | 116.0 |
| (Mother strain in Example 1) | | |
| MO-1 + DSM 33181 | 121.0 | 109.0 |
| Premium | 38.1 | 42.3 |
| Premium + DSM 32589 | 42.7 | 52.2 |
| (Mother strain in Example 1) | | |
| Premium + DSM 33181 | 44.1 | 54.1 |

As will appear from Table 9, the shear stress at both 75.2 Hz and 300 Hz were significantly increased for the culture blend comprising DSM 33181 and for the culture blend comprising the sporulation-positive mother strain DSM 32589 as compared to the starter culture with no Bacillus strain.

Deposits and Expert Solution

The strain Bacillus subtilis subsp. natto deposited at the Leibniz Institute DSMZ-German Collection of Microorganisms and Cell Culture (DSMZ), Inhoffenstr. 7B, 38124 Braunschweig, Germany on Aug. 16, 2017 under the accession number DSM 32588.

The strain Bacillus subtilis subsp. natto deposited at the Leibniz Institute DSMZ-German Collection of Microorganisms and Cell Culture (DSMZ), Inhoffenstr. 7B, 38124 Braunschweig, Germany on Aug. 23, 2017 under the accession number DSM 32606.

The strain Bacillus subtilis subsp. natto deposited at the Leibniz Institute DSMZ-German Collection of Microorganisms and Cell Culture (DSMZ), Inhoffenstr. 7B, 38124 Braunschweig, Germany on Aug. 16, 2017 under the accession number DSM 32589.

The strain Bacillus subtilis subsp. natto deposited at the Leibniz Institute DSMZ-German Collection of Microorganisms and Cell Culture (DSMZ), Inhoffenstr. 7B, 38124 Braunschweig, Germany on Aug. 8, 2018 under the accession number DSM 32892.

The strain Bacillus subtilis subsp. natto deposited at the Leibniz Institute DSMZ-German Collection of Microorganisms and Cell Culture (DSMZ), Inhoffenstr. 7B, 38124 Braunschweig, Germany on Aug. 8, 2018 under the accession number DSM 32893.

The strain Bacillus subtilis subsp. natto deposited at the Leibniz Institute DSMZ-German Collection of Microorganisms and Cell Culture (DSMZ), Inhoffenstr. 7B, 38124 Braunschweig, Germany on Aug. 8, 2018 under the accession number DSM 32894.

The strain Bacillus subtilis subsp. natto deposited at the Leibniz Institute DSMZ-German Collection of Microorganisms and Cell Culture (DSMZ), Inhoffenstr. 7B, 38124 Braunschweig, Germany on Aug. 8, 2018 under the accession number DSM 32895.

The strain Bacillus subtilis subsp. natto deposited at the Leibniz Institute DSMZ-German Collection of Microorganisms and Cell Culture (DSMZ), Inhoffenstr. 7B, 38124 Braunschweig, Germany on Jun. 19, 2019 under the accession number DSM 33181.

The strain Bacillus subtilis subsp. natto deposited at the Leibniz Institute DSMZ-German Collection of Microorganisms and Cell Culture (DSMZ), Inhoffenstr. 7B, 38124 Braunschweig, Germany on Jun. 19, 2019 under the accession number DSM 33182.

The deposits have been made under the conditions of the Budapest Treaty on the International Recognition of the Deposit of Microorganisms for the Purposes of Patent Procedure.

The Applicant requests that a sample of the deposited microorganisms should be made available only to an expert approved by the Applicant.

### REFERENCES

[1] Tasneem et al. (2013) Crit Rev Food Sci Nutr., 54(7):869-79.

[2] WO 2011/026863

[3] US 2009/0011081 A1

[4] U.S. Pat. No. 5,077,063

[5] CN 103300147 A

[6] CN 103190478 A

[7] U.S. Pat. No. 3,674,508

[8] WO 2017/005601 A

[9] Mehta et al. (2016), Journal of Animal Science, vol. 94 No. supplement 5, p. 264-265

All references cited in this patent document are hereby incorporated in their entirety by reference.

1. A method for producing a fermented dairy product, comprising:
fermenting a milk substrate with a lactic acid bacterium starter culture, in the presence of at least one Bacillus strain selected from the group consisting of a sporulation-negative Bacillus subtilis subsp. natto strain and a sporulation-negative Bacillus coagulans strain,
wherein a sporulation-negative strain is a strain which forms no spores when assessed by (i) growing the strain overnight in Veal Infusion Broth (VIB) at 37° C., 180 rpm to obtain a culture of the strain, (ii) inoculating 1% of the culture into 50 ml of a standard sporulation inducing medium contained in a 500 ml baffled shake flask to obtain an inoculated medium, (iii) allowing the inoculated medium to grow overnight at 37° C. under shaking at 200 rpm, and (iv) testing for spores the next day.

2. The method according to claim 1, wherein the Bacillus strain is a sporulation-negative mutant of a sporulation-positive mother strain selected from the group consisting of a Bacillus subtilis subsp. natto strain and a sporulation-negative Bacillus coagulans strain.

3. The method according to claim 1, wherein the Bacillus strain is a sporulation-negative mutant of a sporulation-positive mother strain selected from the group consisting of the Bacillus subtilis subsp. natto strain deposited at the Leibniz Institute DSMZ-German Collection of Microorganisms and Cell Culture (DSMZ) under accession number DSM 32588, the Bacillus subtilis subsp. natto strain deposited at the DSMZ under accession number DSM32589, and the Bacillus subtilis subsp. natto strain deposited at the DSMZ under accession number DSM 32606.

4. The method according to claim 1, wherein the sporulation-negative Bacillus subtilis subsp. natto strain is selected from the group consisting of the Bacillus subtilis subsp. natto strain deposited at the DSMZ under accession number DSM 32892, the Bacillus subtilis subsp. natto strain deposited at the DSMZ under accession number DSM 32893, the Bacillus subtilis subsp. natto strain deposited at the DSMZ under accession number DSM 32894, the Bacillus subtilis subsp. natto strain deposited at the DSMZ under accession number DSM 32895 and mutants thereof.

5. The method according to claim 1, wherein the lactic acid bacterium starter culture comprises a Streptococcus thermophilus strain and a Lactobacillus delbrueckii subsp. bulgaricus strain.

6. The method according to claim 5, wherein the fermented dairy product is selected from the group consisting of a set yogurt, a stirred yogurt and drinking yogurt.

7. The method according to claim 1, wherein the lactic acid bacterium starter culture comprises a Lactococcus lactis strain.

8. The method according to claim 7, wherein the fermented dairy product is a mesophilic fermented dairy product selected from the group consisting of sour cream, sour milk, buttermilk, cultured milk, smetana, quark, tvarog, fresh cheese and cream cheese.

9. A sporulation-negative mutant Bacillus strain selected from the group consisting of a Bacillus subtilis subsp. natto strain and a Bacillus coagulans strain, wherein the mutant strain is a sporulation-negative mutant of a sporulation-positive mother strain, wherein a sporulation-negative strain is a strain which forms no spores when assessed by (i) growing the strain overnight in Veal Infusion Broth (VIB) at 37° C., 180 rpm to obtain a culture of the strain, (ii) inoculating 1% of the culture into 50 ml of a standard sporulation inducing medium contained in a 500 ml baffled shake flask to obtain an inoculated medium, (iii) allowing the inoculated medium to grow overnight at 37° C. under shaking at 200 rpm, and (iv) testing for spores the next day.

10. A composition for producing a fermented dairy product, comprising (a) a lactic acid bacterium starter culture, and (b) a sporulation-negative mutant Bacillus strain according to claim 9.

11. A fermented dairy product obtained by the method according to claim 1.

12. A fermented dairy product, comprising (a) a lactic acid bacterium starter culture, and (b) a sporulation-negative mutant Bacillus strain according to claim 9.

13. A method of for increasing one or more of the shear stress, gel stiffness, and gel firmness of a mesophilic fermented dairy product, comprising fermenting a milk substrate with a mesophilic starter culture in the presence of a sporulation-negative mutant Bacillus strain according to claim 9.
